(19) United States
(12) Patent Application Publication
(10) Pub. No.: US 2010/0023034 A1
(43) Pub. Date: Jan. 28, 2010

(54) CLOT RETRIEVAL METHOD AND DEVICE

(75) Inventors: Richard J. Linder, Sandy, UT (US); Daryl R. Edmiston, Draper, UT (US)

Correspondence Address: Holland & Hart LLP/Coherex Medical, 60 East South Temple, Suite 2000, Salt Lake City, UT, 84111, US

(73) Assignee: COHEREX MEDICAL, INC., Salt Lake City, UT (US)

(21) Appl. No.: 12/488,440

(22) Filed: Jun. 19, 2009

Related U.S. Application Data
(60) Provisional application No. 61/074,116, filed on Jun. 19, 2008.

(51) Int. Cl. A61B 17/22; A61F 2/01

(52) U.S. Cl. 606/159; 606/200

(57) ABSTRACT

Devices, systems and methods for retrieving clot material from a vasculature lumen are provided. In one embodiment, a medical device includes a handle, a catheter, a tether member and a clot retrieval element. The catheter extends from the handle and the clot retrieval element is configured to be positioned within a distal portion of the catheter with the tether member extending between the handle and the clot retrieval element. The clot retrieval element is configured to be deployed from within the catheter so that the catheter moves proximally relative to and separate from the clot retrieval element while maintaining attachment to the clot retrieval element via the tether member. Further, the clot retrieval element is configured to be pulled by the tether member to abut against a distal side of a clot with proximal movement of the tether member to pull the clot from the vasculature lumen.

### CROSS-REFERENCE TO RELATED APPLICATIONS

This application claims the benefit of U.S. Provisional Patent Application No. 61/074,116, entitled CLOT RETRIEVAL METHOD AND DEVICE, filed on Jun. 19, 2008, the disclosure of which is incorporated by reference herein in its entirety.

### TECHNICAL FIELD

The present invention relates generally to a catheter system and, more specifically, a catheter system for retrieving clots from the vasculature.

### BACKGROUND

Clot formation within the vasculature can result in various thromboembolic disorders, such as stroke, pulmonary embolism, peripheral thrombosis, artherosclerosis, and the like. Unfortunately, due to unhealthy lifestyles and poor eating habits, clot formation within the vasculature is becoming more common. Further, even if one minds their eating habits and lives a healthy lifestyle, many are still susceptible to these disorders due to their genetic makeup. Thus, it is desirable to eliminate or remove clot formations from within the vasculature to prevent the possibility of such disorders as noted above.

The medical industry has provided various devices and systems, sometimes referred to as embolectomy devices, to remove foreign matter from the vasculature. Many of these devices and systems are catheter based. For example, one catheter based system is described as a device having adhesive disposed thereon. The device is directed to the clot or occlusion and it contacts the clot with an adhesive covered surface of the device. The device is asserted to “stick” to the clot and withdraw the clot along with the catheter and device.

Another device includes a “basket” that is positioned such that the clot or foreign matter is within the interior of the basket. An opening or mouth of the basket is configured to be closed or cinched so as to capture the clot inside of the basket and the basket is then withdrawn from the blood vessel. Other devices may include a macerator that breaks up the clot into smaller particles. The smaller particles may then be captured by use of a filter or by vacuum means.

However, such systems have been found to have various limitations. Often, such structures are either highly complex in their use and implementation. Additionally, many of such structures and rather complex and difficult to manufacture. Moreover, some of these devices and systems lack the ability to sufficiently or effectively remove foreign matter entirely from the vasculature as desired. Further, many systems have been found to be harsh on the inner vessel walls and cause damage to the walls. Some of the known systems introduce additional medical risks to a patient. Therefore, it would be desirable to provide an embolectomy catheter system that is, for example, safe, less complex to use and to manufacture while also being effective in retrieving clot matter from the vasculature system.

### BRIEF SUMMARY OF THE INVENTION

The present invention is directed to medical devices, systems and methods for retrieving clot material from a vasculature lumen. In accordance with one embodiment, a medical device configured to retrieve clot material from a vasculature lumen is provided and includes a handle having a catheter extending therefrom. At least one clot retrieval element is configured to be positioned within a distal portion of a lumen defined through the catheter, the at least one clot retrieval element being operably attached to the handle by way of a tether member extending through the lumen of the catheter. The at least one clot retrieval element is configured to be deployed from within the catheter from a contracted state to an expanded state. When the at least one clot retrieval member is in the expanded state, it conforms to the geometry of the vasculature lumen and applies pressure to the vasculature lumen.

In accordance with another embodiment of the present invention, a method is provided for retrieving a clot from a vasculature lumen. The method includes extending a catheter within a vasculature lumen through a clot and positioning a distal portion of the catheter distally of the clot. At least one clot retrieval element is deployed from the distal portion of the catheter while maintaining attachment to the at least one clot retrieval element via a tethering member extending through the catheter. The at least one clot retrieval element is conformed to a geometry of the vasculature lumen and pressure is applied to a wall of the vasculature lumen by the at least one clot retrieval element. The distal portion of the catheter is displaced proximally with respect to the clot and the at least one clot retrieval element abuts against a distal side of the clot. The clot is pulled proximally through the vasculature lumen with the clot retrieval element abutted against a distal side of the clot.

### BRIEF DESCRIPTION OF THE SEVERAL VIEWS OF THE DRAWINGS

The foregoing and other advantages of the invention will become apparent upon reading the following detailed description and upon reference to the drawings in which:

FIG. 1 is a side view of a medical device system, according to an embodiment of the present invention;

FIG. 1A is a cross-sectional view of a distal portion of the medical device system depicting a clot retrieval element constrained in the distal portion of the medical device system according to one embodiment;

FIG. 1B is a cross-sectional view of the distal portion of the medical device system depicting the clot retrieval element being deployed from the distal portion of the medical device system according to one embodiment;

FIGS. 2A through 2F depict a method for retrieving a clot from a vessel in the vasculature according to an embodiment of the present invention;

FIG. 3 is a front view of a clot retrieval element according to one embodiment of the present invention;

FIG. 3A is a cross-sectional view, taken along section A-A, of the clot retrieval element depicted in FIG. 3;

FIG. 4 is a front view of another embodiment of a clot retrieval element according to an embodiment of the present invention;

FIG. 4A is a cross-sectional view, taken along section A-A of the clot retrieval element of FIG. 4;

FIG. 5 is a perspective view of a clot retrieval element according to another embodiment of the present invention;

FIG. 6 is a perspective view of a clot retrieval element according to an embodiment of the present invention;

FIG. 7 is a cross-sectional view of a clot retrieval element according to another embodiment of the present invention;

FIG. 8 is a perspective view of a clot retrieval element having a cylindrical configuration according to one embodiment of the present invention;

FIG. 9 is a perspective view of a clot retrieval element having a spherical configuration according to another embodiment of the present invention;

FIG. 10 is a perspective view of a clot retrieval element having multiple protrusions extending from the element, according to an embodiment of the present invention; and

FIG. 11 is a side view of a clot retrieval element including multiple clot retrieval portions interconnected to each other with a tethering system according to another embodiment of the present invention.

### DETAILED DESCRIPTION OF THE INVENTION

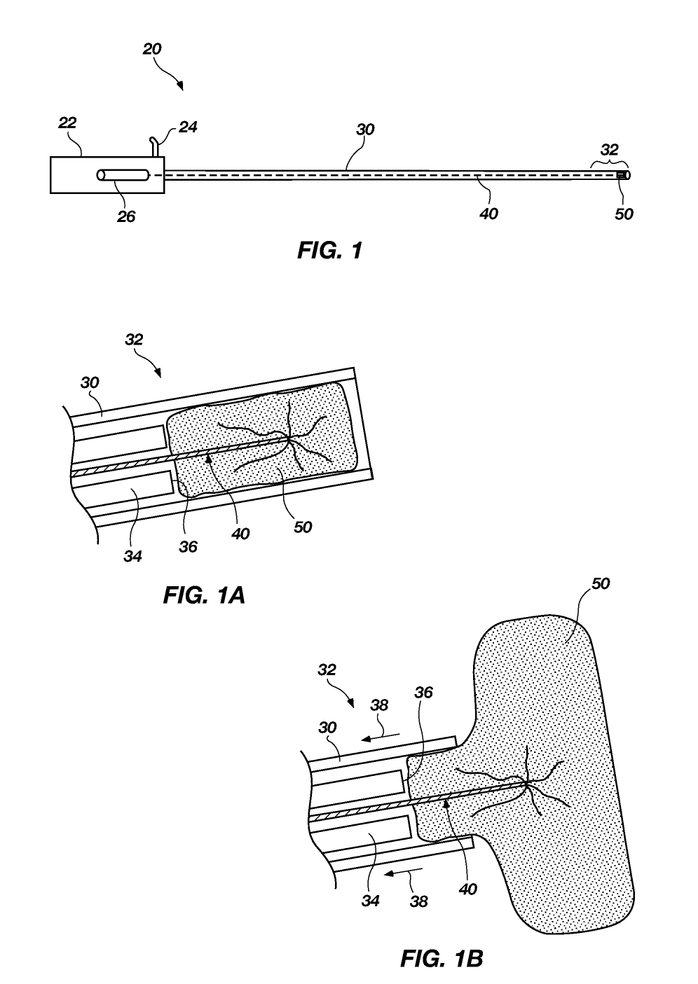

Referring to FIG. 1, a medical device system 20 is shown according to an embodiment of the present invention. Such a medical device system 20 may be employed as an embolectomy catheter. The medical device system 20 includes a handle 22 and a catheter 30 extending distal of the handle. The handle 22 may include a port 24 for passing fluid through the catheter as will be appreciated by those of ordinary skill in the art. The handle 22 may also include an actuator 26 configured to control and manipulate a clot retrieval element 50 disposed in a distal portion 32 of the catheter 30. As such, the actuator 26 is operably interconnected with the clot retrieval element 50 such as through a tethering system or tethering member 40 (shown in dashed lines) extending through catheter 30 between the handle 22 and the clot retrieval element 50. The catheter 30 may be formed from flexible polymeric materials, such as known to those of ordinary skill in the art, that are suitable and configured for delivery within various vessels within the vasculature including, for example, intracranial vessels. In one embodiment, the catheter may be configured to include structure for over-the-wire delivery with a guide wire. In another embodiment, the catheter can include suitable structure to be delivered via a guide wire employing, what is commonly termed as, rapid exchange. In still another embodiment, the catheter may be sized and configured as a micro-catheter for suitable delivery within an intracranial vessel. Such micro-catheter may be guided through the vasculature by a suitable sized guide wire as known in the art.

FIGS. 1A and 1B disclose enlarged cross-sectional views of the distal portion 32 of the catheter 30, depicting the clot retrieval element 50 positioned within the catheter (FIG. 1A) and being deployed from the catheter (FIG. 1B). In particular, the clot retrieval element 50 is sized and configured to be positioned within the distal portion 32 of the catheter 30 in a compact and constrained configuration (see FIG. 1A). With such an arrangement, the catheter 30 may include an inner lumen 34 extending at least partially through a length of the catheter so that a lumen end 36 of the inner lumen 34 is positioned proximally adjacent the clot retrieval element 50. Such lumen end 36 is sized and configured to act with the actuator 26 (FIG. 1) to provide a back-stop for the clot retrieval element 50. Further, the tethering member 40, interconnected to the handle 22 (FIG. 1), may extend through the inner lumen 34. Such tethering member 40 can be operably interconnected with, or otherwise attached to the clot retrieval element 50. The clot retrieval element 50 can be moved from the catheter 30 with the actuator 26 by moving the catheter 30 in a proximal direction, as indicated by arrows 38, while the inner lumen 34 maintains a substantially fixed position (relative to the catheter 30) to act as a back-stop to the clot retrieval element 50. In another embodiment, the inner lumen 34 can be moved in a distal direction while the catheter 30 is moved proximally or remains in a fixed position. Thus, the clot retrieval element 50 may be deployed from the distal portion 32 of the catheter 30 by relative movement of the catheter 30 and the inner lumen 34.

As the clot retrieval element 50 is deployed and becomes exposed from the catheter 30, the clot retrieval element 50 self expands to an enlarged or expanded configuration. Such a clot retrieval element 50 expands to a size and configuration that enables it to conform to the dimension of the vasculature lumen its being deployed within. Thus, the clot retrieval element 50 may expand to effect contact with the entire circumference of a vasculature lumen at a given location. In the expanded configuration, the clot retrieval element 50 can remain interconnected to the medical device system 20 via the tethering member 40.

Further, the clot retrieval element 50 is configured to provide an expansion force or a radial outward force against the vessel wall while in the expanded configuration. This force may be applied radially to the entire circumference of the vasculature lumen at a given cross-section thereof. The engagement of the clot retrieval element 50 may be effected for a desired distance along the length of the vessel wall. For example, in one embodiment, it may be desirable to engage the vessel wall about its circumference and for a length of at least 1 millimeter (mm) or at least 2 mm. In another embodiment, it may be desirable to engage the vessel wall for a length of between approximately 3 mm and approximately 7 mm. In another embodiment, it may be desirable to engage the wall for a length that is approximately equal to a diameter of the vessel in which the clot retrieval element is to be disposed.

The clot retrieval element 50 may be formed from a polymeric material or any other suitable self expanding material that is generally soft and supple and that will not cause damage to the vessel walls. Further, in one embodiment, the clot retrieval element 50 may include a porous material, such as foam, mesh, fabric, felt and/or any other suitable material having a porous structure that will facilitate the clot retrieval element to be placed in compact configuration and self expand to an enlarged or expanded configuration that substantially conforms to the dimension of, for example, the vessel the clot retrieval element is disposed within. Some example materials that the clot retrieval element may be formed from include, without limitation, polyurethane, polytetrafluoroethylene (PTFE—sometimes marketed under the trademark Teflon®), expanded PTFE (ePTFE), polyester, silicon, polyethylene terephthalate (PET—sometimes marketed under the trademark Dacron®, composites or combinations thereof, or any other suitable material known to one of ordinary skill in the art. It is also contemplated that the clot retrieval element 50 can include a marker by, for example, impregnating or coating the clot retrieval element to facilitate viewing the placement and position of the clot retrieval element within the vasculature as well as within the medical device system utilizing conventional imaging techniques. The marker can also be provided with the tethering member. Such marker can be formed from a radio-opaque material, such as tantalum, gold, platinum or alloys thereof, or any other suitable radio-opaque material, such as barium sulfate, as known in the art.

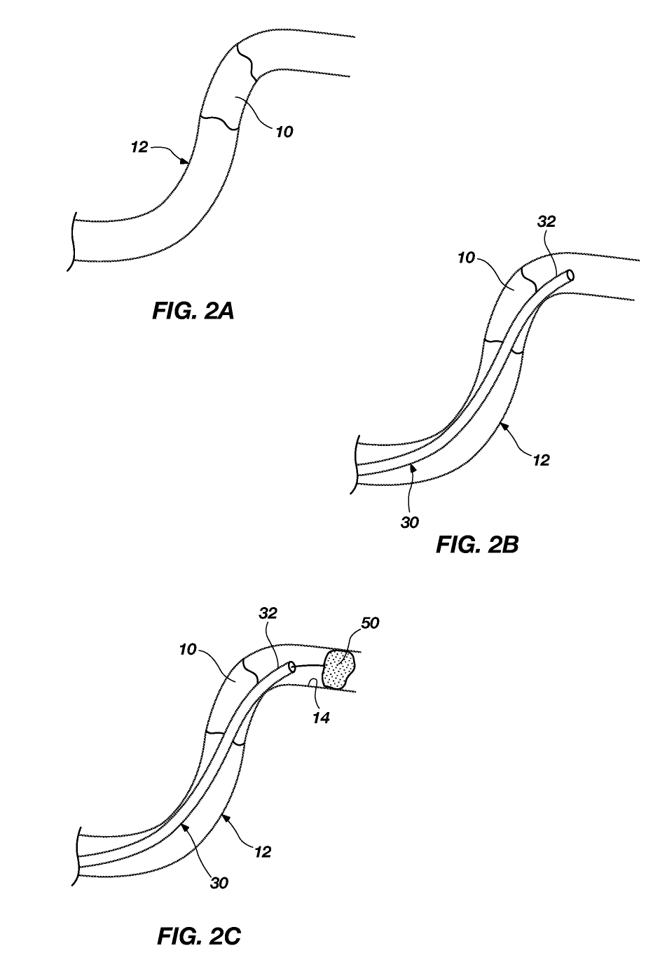

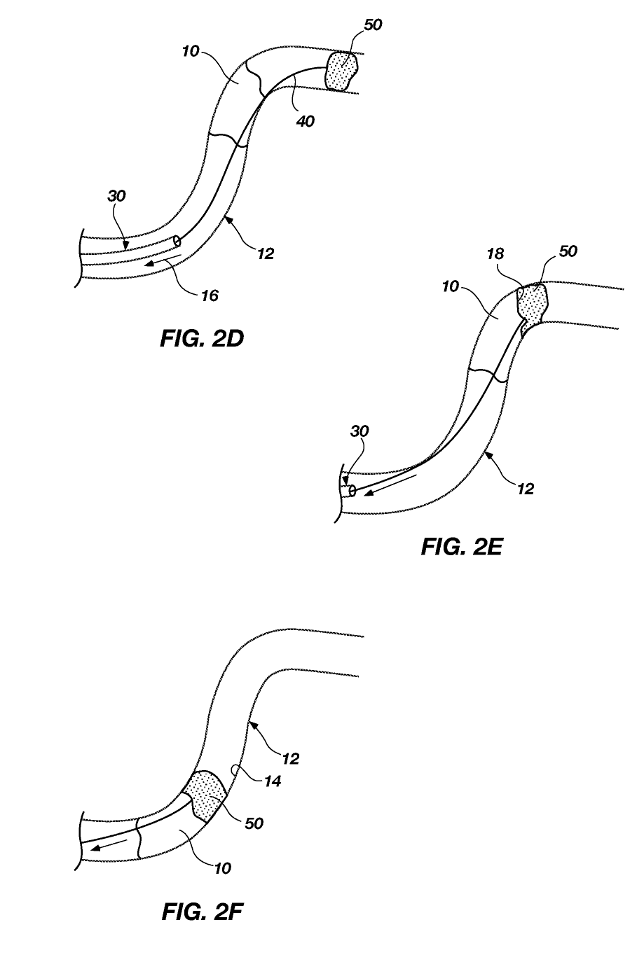

Referring now to FIGS. 2A through 2F a method is described for retrieving a clot 10 from a vessel 12. FIG. 2A shows an example of a vasculature lumen or vessel 12 with a clot 10 formed therein. Such a vasculature lumen may be an intracranial vessel, a peripheral vessel, or any other vasculature lumen having a clot formed therein. FIG. 2B depicts the catheter 30 of the medical device system 20 of the present invention positioned within the vessel 12 and the distal portion 32 of the catheter 30 extending distally beyond the clot 10. Positioning the catheter 30 distally of the clot 10 may be accomplished, for example, utilizing a micro-catheter for intracranial applications, or utilizing over-the-wire or rapid-exchange techniques for the same or for other applications. Of course, as known to one of ordinary skill in the art, the catheter 30 may include various structures associated with the employment of over-the-wire or rapid exchange techniques, or it may employ structures and features associated with micro-catheters.

Once in the distal position (e.g., as shown in FIG. 2B), the user can then deploy the clot retrieval element 50 from the catheter 30 as depicted in FIG. 2C (See also FIG. 1B). Once deployed, the clot retrieval element 50 expands from the constrained compact configuration (as contained within the catheter 30) to an expanded configuration such that the clot retrieval element 50 contacts the wall 14 of the vessel 12. Moreover, as noted above, the clot retrieval element 50 expands so as to conform with the size and shape of the vessel 12 and may press against the wall 14 with a desired magnitude of radial force or pressure. As depicted in FIG. 2D, the catheter 30 may be displaced proximally, as depicted by arrow 16, with respect to the clot 10 while maintaining attachment to the clot retrieval element 50 with the tethering member 40. As the catheter 30 continues to be withdrawn, moving proximally, the clot retrieval element 50 also moves proximally to abut against a distal side 18 of the clot 10 as depicted in FIG. 2E.

As depicted in FIG. 2F, the catheter 30 (not shown) and clot retrieval element 50 continue to move proximally to ultimately withdrawn the clot from the vessel 12. During removal of the clot 10, the clot retrieval element 50 is maintained on a distal side 18 of the clot 10 as the catheter 30 is withdrawn from the vessel 12 due to the clot retrieval element conforming to the blood vessel and effectively filling the cross-sectional area of the vessel on the distal side of the clot 10 by application of a continual outward force against the wall 14 of the vessel 12. In this manner, the clot 10 is withdrawn from the vessel 12 as the catheter 30 and clot retrieval element 50 are also withdrawn from the vessel 12. It is also contemplated that as the clot 10 is withdrawn from a vessel 12, the clot 10 may be pulled toward the catheter it may be suctioned into and through the catheter. It is noted that, in the embodiment shown and described with respect to FIGS. 2A-2F, normal blood flow within the vessel 12 is along the catheter 30 towards the deployed clot retrieval element 50 (i.e., opposite to the direction indicated by 16).

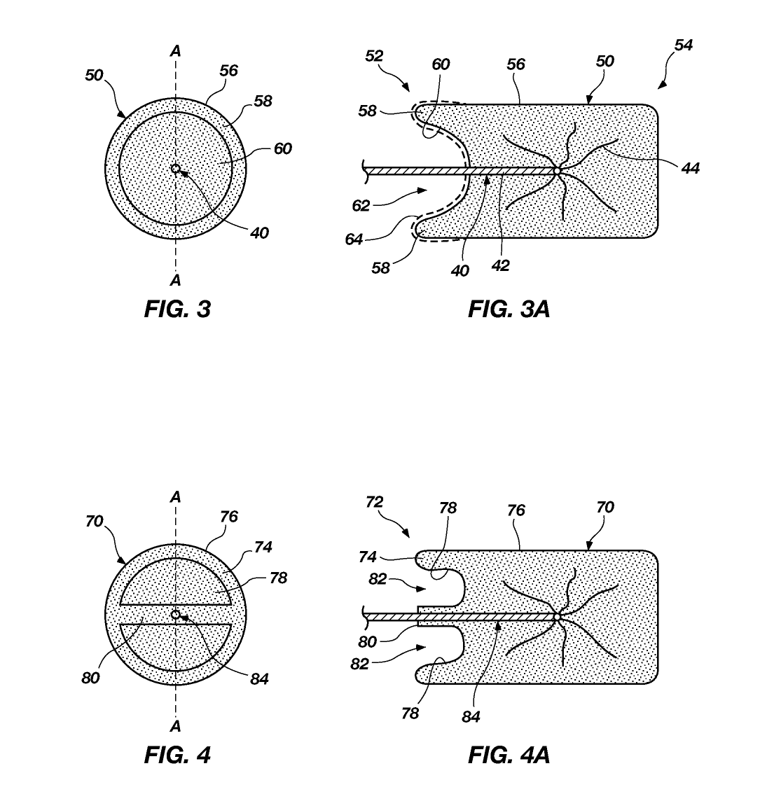

FIGS. 3 and 3A disclose an embodiment of a clot retrieval element 50 with a tethering member 40 attached thereto. Such a clot retrieval element 50 may be substantially cylindrical in shape (in its expanded form) with a proximal side 52, a distal side 54 and an outer radial surface 56. The proximal side 52 may include a rim portion 58 located peripherally between the radial surface 56 and a cavity surface 60 at the proximal side 52. The cavity surface 60 can extend from the rim portion 58 in a concave configuration within the proximal side 52 to define a cavity 62. Further, the proximal side 52 of the clot retrieval element 50 can include the tethering member 40 centrally extending into the cavity surface 60. The cavity 62 of the clot retrieval element 50 may be sized and configured to assist in capturing and holding a clot as the clot retrieval element 50 is pulled proximally through the vasculature. As previously set forth, the clot retrieval element 50 can be formed from a polymeric material, such as foam. As such, in one embodiment, the polymeric material or foam can be formed with a higher density at the proximal side to assist in supporting the rim portion 58 of the clot retrieval element 50. One method for forming the clot retrieval element 50 may include a molding process as known by those of ordinary skill in the art. In another embodiment, the proximal side 52 of the clot retrieval element 50 may include one or more layers 64 (shown in dashed lines) of a strengthening material to increase the structural support of the rim portion 58 and cavity surface 60 and substantially prevent potential collapsing of the rim portion 58 when pulling the clot retrieval element 50. In one embodiment, the added layer 64 may include a polymer film laminated to the proximal side 52. Such a material may include, for example, ePTFE or polyurethane (PU). In another embodiment the proximal side 52 may be stiffened by, for example, the selective addition of an adhesive to the matrix material (e.g., foam) forming the body of the clot retrieval element 50. Such an adhesive may include, for example, an acrylic or silicone based adhesive material.

As previously discussed, the clot retrieval element 50 includes a tethering member 40 centrally extending into the proximal side 52 of the clot retrieval element 50 and within the cavity surface 60. In one embodiment, the tethering member 40 may include a line portion 42 and multiple end portions 44 extending from the line portion 42. The line portion 42 includes a proximal end (not shown) and a distal end 46, the proximal end being operably interconnected to the handle (see FIG. 1) and the distal end extending within the clot retrieval element 50. In one embodiment, the line portion 42 may include multiple lines or filaments wound together or braided to comprise the line portion. The multiple lines, at the distal end 46, may be separated to comprise the multiple end portions 44 each extending in different directions within the body of the clot retrieval element 50. Such multiple end portions 44 can be configured to be positioned throughout the clot retrieval element 50 in random positions or in predetermined positions with many, if not all, extending at an angle relative to the line portion 42. With this arrangement, the multiple end portions 44 extending throughout the clot retrieval element 50 are configured to support and maintain attachment to the clot retrieval element in the expanded configuration while applying a force to the clot retrieval element to pull it through the vessel both before and after engaging the clot.

FIGS. 4 and 4A disclose another embodiment of a clot retrieval element 70. The clot retrieval element is generally similar to that which is described in the previous embodiment. However, in the presently considered embodiment, the clot retrieval element 70 may be configured to define multiple cavities 82 in a proximal side 72 of the clot retrieval element 70. In particular, the proximal side 72 of the clot retrieval element 70 may include a rim portion 74 located peripherally between a radial surface 76 and a cavity surface 78. The cavity surface 78 may include one or more cross members 80 to help define the multiple cavity portions 82 within the proximal side 72 of the clot retrieval element 70. A tethering member 84 may extend through the cross member 80 which may provide added strength and support with regard to the interconnection of the tethering member 84 and the clot retrieval element 70. With this arrangement, the clot retrieval element 70 can abut against a distal side of a clot and, as the clot retrieval element 70 is pulled against the clot, at least a portion of the clot can be captured within the multiple cavity portions 82 defined in the clot retrieval element 70. As in the previous embodiment, the proximal side 72 of the clot retrieval element 70 can be formed of a higher density of foam than that of the distal side of the clot retrieval element and/or the proximal side may include one or more layers to assist in supporting the rim portion and cross member (or members) of the clot retrieval element 70 when being employed to capture a clot.

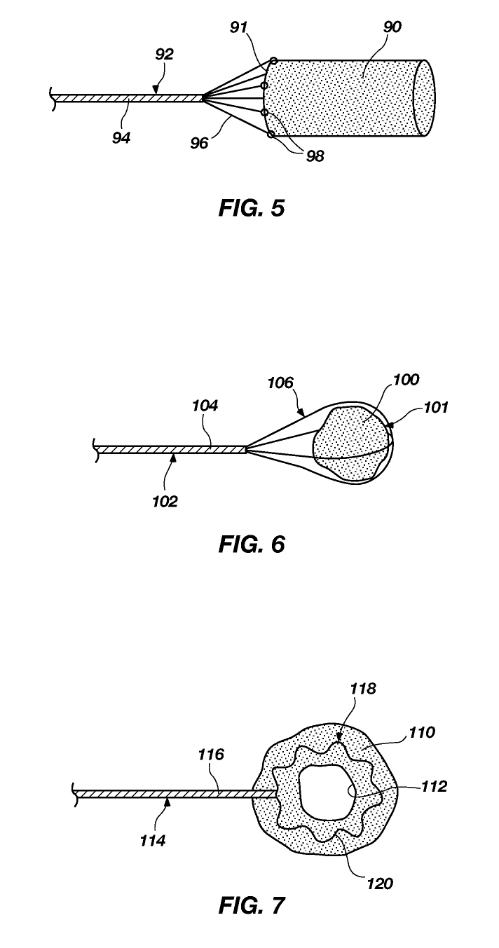

Referring now to FIG. 5, another embodiment of a clot retrieval element 90 coupled with a tethering member 92 is shown. In this embodiment, the tethering member 92 includes a line portion 94 with multiple end portions 96 extending from the line portion. The line portion 94 may include, for example, multiple lines wound together and, at a distal end of the line portion 94, the multiple end portions 96 may be configured to extend distally to attach to the proximal side 91 of the clot retrieval element 90 at discrete attachment points 98. The attachment points 98 may be reinforced to withstand desired forces when the tethering member is pulled, thus applying forces to each of the attachment points 98 through the end portions 96.

FIG. 6 discloses another embodiment of the tethering member 102 operatively interconnected to a clot retrieval element 100. In particular, the tethering member 102 may include a line portion 104 and a holding portion 106 comprising multiple loop portions. The line portion 104 can include, for example, multiple lines wound together while the holding portion 106 may include the multiple lines in a non-wound configuration such that the multiple lines extend externally around a distal portion 101 of the clot retrieval element 100 to hold or encompass the clot retrieval element within the wrapped multiple lines extending therearound. In one embodiment, attachment points may be disposed at one or more positions about the clot retrieval element 100 to help maintain spacing of the individual lines of the holding portion.

Referring to FIG. 7, a cross-sectional view of a tethering member 112 attached to a clot retrieval element 110 according to another embodiment is shown. The clot retrieval element includes a hollow portion 112 or an evacuated volume defined therein. In this embodiment, the tethering member 114 may include a line portion 116 and a holding portion 1 18. Similar to the previous embodiments, the line portion 116 may include multiple lines wound together to form the line portion 116. The holding portion 118 extends from the line portion 116 and also can include the multiple lines therewith. The individual lines of the holding portion 118 may extend internally through the clot retrieval element 110 in loop configurations such that they return back to the line portion 116. Such loops can also include undulations 120 to support and stabilize the clot retrieval element 110. As depicted, the holding portion 118 can extend through the clot retrieval element 110 by extending around the hollow portion 112 defined in the clot retrieval element 110. In one embodiment, the line or lines of the holding portion 118 are coupled at one end or at both ends to the line portion 116 such as by knots or other fastening mechanisms or techniques. In another embodiment, the line or lines of the holding portion 118 may be unitary extensions of the line portion 116 at one end or both ends of the line portions.

The tethering system or tethering member used in various embodiments of the present invention may be formed from any suitable metallic or polymeric material. In one embodiment, the tethering member may be formed from one or more wires of a metallic material, such as Nitinol, or any other suitable metallic material. Such wire or wires can also be employed to provide a modulation to the radial force of the clot retrieval element when expanded. This may be accomplished, for example, by varying the diameter of the wire along its length. This may also be accomplished by utilizing a shape memory alloy, such as Nitinol, which is heat set so that the unconstrained profile provides a desired expansive force in contacting a vessel wall. Yet another example of accomplishing modulation of the radial force might include using “wires” that are constructed of tube material and are strategically cut to exhibit variable strut widths. Other means of providing a modulation of the radial force may also be used, and such examples are not intended to be limiting.

In another embodiment, the tethering member can be formed from a high tensile fiber of, for example, a single line, or fiber bundle, braid or multiple bundles braided, and formed from a any suitable polymeric material, such as PET, providing sufficient tensile strength suitable to act as a tether and interconnection between the clot retrieval element and the handle to maintain attachment to the clot retrieval element while pulling the clot retrieval element through the vasculature to withdraw the clot therefrom. In any case, the tethering member can extend between the clot retrieval element and the handle and can include additional components as known to one of ordinary skill in the art to control and enable maneuverability, for example, via the handle. Further, the tethering member can be attached to the clot retrieval element by other suitable techniques such as by molding the tethering member into the clot retrieval element.

FIG. 8 discloses one embodiment of a clot retrieval element 130, depicted in a non-constrained, expanded configuration. In particular, the clot retrieval element 130 can be substantially cylindrical in shape with a proximal surface 132 and a distal surface 134 generally being flat. As previously set forth, the clot retrieval element 130 can be made from a polymeric and/or metallic material, such as foam, to provide the characteristics that employ both the compact and expanded configurations while providing sufficient strength to engage and pull a clot through the vasculature and while not causing damage to the vasculature during the process. Further, as shown in dashed lines, the clot retrieval element 130 can include a hollow portion 136 to minimize the size or volume of the compact configuration while maximizing the size or volume of the expanded configuration of the clot retrieval element 130.

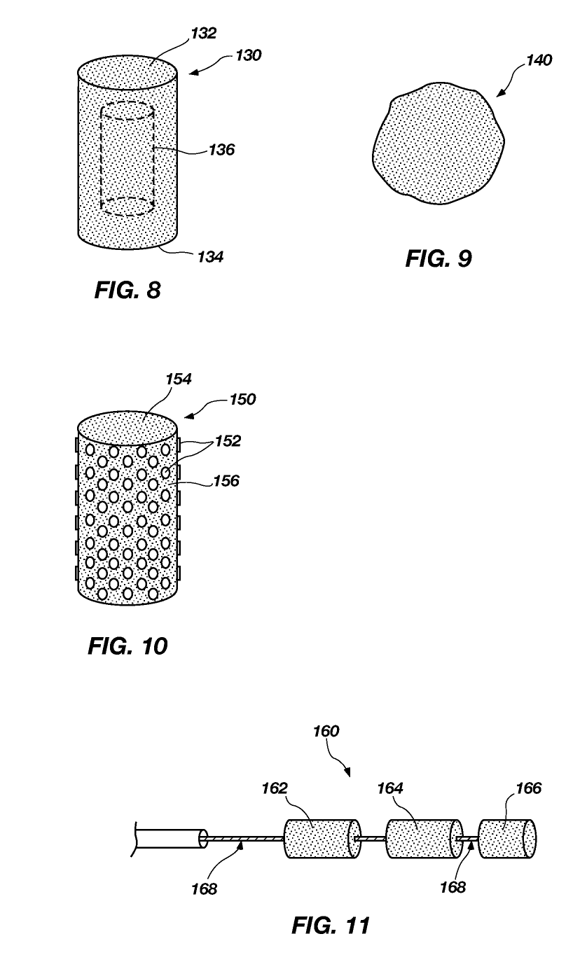

FIG. 9 is similar to the embodiment depicted in FIG. 8, except in this embodiment the clot retrieval element 140 is substantially spherical in shape when in a non-constrained fully expanded configuration. It should be noted that when expanded in a vessel, the clot retrieval element 140 will preferably be in a substantially cylindrical shape (or at least having a portion that is substantially cylindrical, with potentially domed end portions) due to the constraints of the vessel dimension and resistance to any radial pressure applied by the clot retrieval element 140. In this embodiment, the clot retrieval element 140 can also include a hollow portion (not shown) to maximize the ratio of expansion volume to contraction volume.

FIG. 10 discloses another embodiment of the clot retrieval element 150, also depicted in a non-constrained, expanded configuration, that includes multiple protrusions 152 extending therefrom to provide a textured surface to the clot retrieval element. In this embodiment, the multiple protrusions 152 can extend from the radial surface 156 of the clot retrieval element 150, wherein the proximal side 154 may not include the protrusions. Such multiple protrusions 152 can be employed to scrub and catch any residual clot matter located along the vessel wall as the clot retrieval element 150 is being pulled against the clot and through the vasculature. Such protrusions can be configured to be supple so as to not damage the vessel wall. As such, the multiple protrusions 152 scrub the vessel wall of any clot residue left on the vessel wall as the majority of the clot is being withdrawn from the vessel. In one embodiment, the protrusions 152 may be formed of the same material as the body of the clot retrieval element 150, and may be formed, for example, by molding the clot retrieval element to exhibit such protrusions 152.

Referring now to FIG. 11, there is disclosed another embodiment of a clot retrieval element 160 that includes multiple clot retrieval portions or discrete bodies. For example, the clot retrieval element 160 of this embodiment can include a proximal clot retrieval portion 162 (or discrete body), an intermediate clot retrieval portion 164 (or discrete body) and a distal clot retrieval portion 166 (or discrete body). Such clot retrieval portions can be separated from each other while remaining coupled to each other by a tethering member 168 extending between each clot retrieval portion. In other words, spaces or gaps may be defined between adjacent clot retrieval portions.

The clot retrieval portions 162, 164, and 166 may be lined in a sequence with multiple clot retrieval portions (two or more). The clot retrieval portions can be sized and configured similar to the various embodiments previously described. The more distal clot retrieval portions may act to catch and retrieve any clot matter that was not captured by the more proximal clot retrieval portions. Thus, the additional clot retrieval portions provide redundancy and help to ensure the most complete removal of clot matter from the vasculature. This may occur when the system is being withdrawn so that the additional clot retrieval portions catch potential clot matter that may have become loose and migrated after the proximal-most clot retrieval portion bypasses the original position of the clot. In one embodiment, the various clot retrieval portions can be substantially similar in size, shape and material. In another embodiment, the clot retrieval portions can include variations in size, shape and material to effect slightly different functions. For example, in one embodiment the proximal-most clot retrieval portion 162 and the distal-most clot retrieval portion 166 can be configured similar to the clot retrieval element disclosed in FIGS. 3 and 3A, while a mid clot retrieval portion 164 may be configured similar to the clot retrieval element with protrusions depicted in FIG. 10. In this manner, the clot retrieval element 160 depicted in FIG. 11 can maximize the effectiveness in removing or retrieving a clot from the vasculature.

It is also noted that various features described with reference to one specific embodiment may be employed in other embodiments. While the invention may be susceptible to various modifications and alternative forms, specific embodiments have been shown by way of example in the drawings and have been described in detail herein. However, it should be understood that the invention is not intended to be limited to the particular forms disclosed. Rather, the present invention includes all modifications, equivalents, and alternatives falling within the spirit and scope of the invention as defined by the following appended claims.

What is claimed is:

1. A medical device configured to retrieve clot material from a vasculature lumen, comprising:
a handle having a catheter extending therefrom;
at least one clot retrieval element configured to be positioned within a distal portion of a lumen defined through the catheter, the at least one clot retrieval element operably attached to the handle with a tether member extending through the lumen of the catheter;
wherein the at least one clot retrieval element is configured to be deployed from within the catheter from a contracted state to an expanded state such that, when in the expanded state, the at least one clot retrieval element conforms to the geometry of the vasculature lumen and applies pressure to the vasculature lumen.

2. The medical device of claim 1, wherein the at least one clot retrieval element comprises a foam material.

3. The medical device of claim 1, wherein the at least one clot retrieval element includes at least a portion that is substantially cylindrical when in the expanded state.

4. The medical device of claim 3, wherein the at least one clot retrieval element includes a proximal end, a radial wall and a distal end, and wherein at least a portion of the proximal end exhibits a concave surface.

5. The medical device of claim 4, wherein a volume of material at the proximal end is exhibits a higher density than a volume of material at the distal end.

6. The medical device of claim 4, wherein at least one clot retrieval method further comprises at least one cross member extending across a portion of the proximal end.

7. The medical device of claim 4, wherein at least one clot retrieval element further comprises a reinforcing layer disposed on the proximal end.

8. The medical device of claim 1, wherein the tether member includes a plurality of lines, wherein ends of the plurality of lines are disposed within an interior portion of the at least one clot retrieval element.

9. The medical device of claim 8, wherein the ends of the plurality of lines are each positioned at different locations within the interior portion of the at least one clot retrieval element.

10. The medical device of claim 1, wherein the at least one clot retrieval element includes a body having a substantially hollow area defined therein.

11. The medical device of claim 1, wherein at least one clot retrieval element includes a plurality of discrete clot retrieval elements coupled with the tether member and spaced apart from one another.

12. The medical device of claim 11, wherein at least one of the plurality of clot retrieval elements includes a plurality of protrusions on a radial surface thereof.

13. The medical device of claim 11, wherein each of the plurality of clot retrieval elements are configured substantially identical to one another.

14. The medical device of claim 1, wherein the catheter is configured as a micro-catheter.

15. A method of retrieving a clot from a vasculature lumen, the method comprising:
extending a catheter within a vasculature lumen through a clot;
positioning a distal portion of the catheter distally of the clot;
deploying at least one clot retrieval element from the distal portion of the catheter while maintaining attachment to the at least one clot retrieval element via a tethering member extending through the catheter;
conforming the at least one clot retrieval element to a geometry of the vasculature lumen and applying pressure to a wall of the vasculature lumen with the at least one clot retrieval element;
displacing the distal portion of the catheter proximally with respect to the clot;
abutting the at least one clot retrieval element against a distal side of the clot; and
pulling the clot proximally through the vasculature lumen with the clot retrieval element abutted against a distal side of the clot.

16. The method according to claim 15, further comprising maintaining pressure on the vasculature lumen while moving the distal portion of the catheter proximally with respect to the clot.

17. The method according to claim 15, further comprising maintaining pressure on the vasculature lumen while pulling the clot proximally through the vasculature lumen.

18. The method according to claim 15, wherein deploying at least one clot retrieval element from the distal portion of the catheter further comprising deploying a body comprising a foam material from the distal portion of the catheter.

19. The method according to claim 15, wherein deploying at least one clot retrieval element from the distal portion of the catheter includes deploying a plurality of discrete, spaced apart clot retrieval elements coupled together by the tethering member.

20. The method according to claim 19, wherein deploying a plurality of discrete, spaced apart clot retrieval elements includes deploying a plurality of substantially identical clot retrieval elements.

21. The method according to claim 19, wherein deploying a plurality of discrete, spaced apart clot retrieval elements includes deploying at least one clot retrieval element having a plurality of protrusions on a radial surface thereof.

22. The method according to claim 15, wherein deploying at least one clot retrieval element from the distal portion of the catheter includes deploying a clot retrieval element having a substantially convex surface in at least a portion of a proximal end of the clot retrieval element.

23. The method according to claim 15, wherein deploying at least one clot retrieval element from the distal portion of the catheter includes deploying a clot retrieval element having a volume of material at a proximal end thereof exhibiting a first density and a volume of material at a distal end thereof exhibiting a second density, the first density being greater than the second density.
